(19) United States
(12) Patent Application Publication
(10) Pub. No.: US 2025/0332292 A1
(43) Pub. Date: Oct. 30, 2025

(54) IN VIVO CROSSLINKABLE HYDROGELS WITH COVALENTLY ATTACHED DYE FOR MEDICAL APPLICATIONS

(71) Applicant: Boston Scientific Scimed, Inc., Maple Grove, MN (US)

(72) Inventors: Liming Crandall, Lino Lakes, MN (US); Rick William Dorn, Plymouth, MN (US); Joseph Thomas Delaney, JR., Minneapolis, MN (US)

(73) Assignee: Boston Scientific Scimed, Inc., Maple Grove, MN (US)

(21) Appl. No.: 19/187,248

(22) Filed: Apr. 23, 2025

Related U.S. Application Data
(60) Provisional application No. 63/638,293, filed on Apr. 24, 2024.

(51) Int. Cl. A61K 49/00

(52) U.S. Cl. CPC A61K 49/006; A61K 49/0073

(57) ABSTRACT

In some aspects, the present disclosure pertains to systems that comprise (a) one or more reactive multi-arm polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule. Other aspects the present disclosure pertain to methods of treatment using such systems and dye-containing hydrogel compositions comprising crosslinked reaction products of such systems.

### CROSS-REFERENCE TO RELATED APPLICATIONS

This application claims the benefit of U.S. Provisional Patent Application Ser. No. 63/638,293 filed on Apr. 24, 2024, the disclosure of which is incorporated herein by reference.

### FIELD

The present disclosure relates to dye-containing hydrogels and to crosslinkable systems for forming dye-containing hydrogels, among other aspects. The dye-containing hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triliodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups rapidly react with the trilysine crosslinker in vivo to form a hydrogel. The hydrogels breakdown in-vivo over the course of ca. 6-9 months. The breakdown occurs primarily through the hydrolysis of the ester linkages on the glutarate groups.

However, not all applications require hydrogels to be visible under X-ray irradiation. For example, various applications including the use of hydrogels as lifting agents, can be performed with colored hydrogels.

### SUMMARY

In various aspects, the present disclosure provides hydrogels that are colored in the visible region of the electromagnetic spectrum.

As used herein, a “hydrogel,” also referred to herein as a “crosslinked hydrogel,” is a crosslinked polymer that contains water or can absorb water but does not dissolve when placed in water.

In some aspects, the present disclosure pertains to systems that comprise (a) one or more reactive multi-arm polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule.

In some embodiments, the system comprises a plurality of the one or more reactive multi-arm polymers, wherein a first fraction of the plurality of the one or more reactive multi-arm polymers comprise the covalently attached dye molecule, and a second fraction of the plurality of the one or more reactive multi-arm polymers do not comprise the covalently attached dye molecule. In some of these embodiments, the covalently attached dye molecule is covalently attached to the first fraction of the plurality of the one or more reactive multi-arm polymers through an amide group, the covalently attached dye molecule is covalently attached to the first fraction of the plurality of the one or more reactive multi-arm polymers through an ester group, the covalently attached dye molecule is covalently attached to the first fraction of the plurality of the one or more reactive multi-arm polymers through a triazine group, the covalently attached dye molecule is covalently attached to the first fraction of the plurality of the one or more reactive multi-arm polymers through an amine group, or the covalently attached dye molecule is covalently attached to the first fraction of the plurality of the one or more reactive multi-arm polymers through an ether group. In some of these embodiments, the covalently attached dye molecule is covalently attached to the first fraction of the plurality of the one or more reactive multi-arm polymers through an amide group and an ester group, the covalently attached dye molecule is covalently attached to the first fraction of the plurality of the one or more reactive multi-arm polymers through two ester groups, or the covalently attached dye molecule is covalently attached to the first fraction of the plurality of the one or more reactive multi-arm polymers through a triazine group and an ester group.

In some embodiments, the system comprises the system comprises a plurality of the one or more reactive multifunctional compounds, wherein a first fraction of the plurality of the one or more reactive multifunctional compounds comprise the covalently attached dye molecule, and wherein a second fraction of the plurality of the one or more reactive multifunctional compounds do not comprise the covalently attached dye molecule. In some of these embodiments, the covalently attached dye molecule is covalently attached to the first fraction of the plurality of the one or more reactive multifunctional compounds through an amide group, the covalently attached dye molecule is covalently attached to the first fraction of the plurality of the one or more reactive multifunctional compounds through an ester group, the covalently attached dye molecule is covalently attached to the first fraction of the plurality of the one or more reactive multifunctional compounds through a triazine group, the covalently attached dye molecule is covalently attached to the first fraction of the plurality of the one or more reactive multifunctional compounds through an amine group, or the covalently attached dye molecule is covalently attached to the first fraction of the plurality of the one or more reactive multifunctional compounds through an ether group. In some of these embodiments, the covalently attached dye molecule is covalently attached to the first fraction of the plurality of the one or more reactive multifunctional compounds through an amide group and an ester group, the covalently attached dye molecule is covalently attached to the first fraction of the plurality of the one or more reactive multifunctional compounds through two ester groups, or the covalently attached dye molecule is covalently attached to the first fraction of the plurality of the one or more reactive multifunctional compounds through a triazine group and an ester group.

In some embodiments, which can be used in conjunction with the above embodiments, the first fraction ranges from 0.00001 mol % to 0.1 mol % and the second fraction ranges from 99.9 mol % to 99.99999 mol %.

In some embodiments, which can be used in conjunction with the above embodiments, the weight of the first fraction ranges from 0.1 ppm to 1000 ppm of the total weight of the first and second fractions.

In some embodiments, which can be used in conjunction with the above aspects and embodiments, the core region comprises a polyol residue.

In some embodiments, which can be used in conjunction with the above aspects and embodiments, the hydrophilic polymer segments are selected from poly(alkylene oxide) segments, polysaccharide segments, polyoxazoline segments, polydioxanone segments, polypeptide segments, and polyvinyl alcohol segments.

In some embodiments, which can be used in conjunction with the above aspects and embodiments, each of the plurality of hydrophilic polymer segments contains between 10 and 1000 monomer residues.

In some embodiments, which can be used in conjunction with the above aspects and embodiments, the plurality of first reactive moieties comprise a cyclic imide ester group and the plurality of second reactive moieties comprise a primary amine, thiol or hydroxyl group, or the plurality of first reactive moieties comprise a primary amine, thiol or hydroxyl group and the plurality of second reactive moieties comprise a cyclic imide ester group.

In some embodiments, which can be used in conjunction with the above aspects and embodiments, the plurality of first reactive moieties comprise a strained alkyne group and the plurality of second reactive moieties comprise an azide group, or the plurality of first reactive moieties comprise an azide group and the plurality of second reactive moieties comprise a strained alkyne group.

In some embodiments, which can be used in conjunction with the above aspects and embodiments, the plurality of first reactive moieties comprise a strained alkene group and the plurality of second reactive moieties comprise a tetrazine group, or the plurality of first reactive moieties comprise a tetrazine group and the plurality of second reactive moieties comprise a strained alkene group.

In some embodiments, which can be used in conjunction with the above aspects and embodiments, the system further comprises a delivery device.

In other aspects, the present disclosure pertains to methods of treatment comprising administering to a subject a mixture that comprises the one or more reactive multi-arm polymers, the one or more reactive multifunctional compounds, and the covalently attached dye molecule of any of the above aspects and embodiments under conditions such that the one or more reactive multi-arm polymers and the one or more reactive multifunctional compounds covalently crosslink after administration to form a hydrogel, wherein the covalently attached dye molecule is covalently attached to the hydrogel.

In some embodiments, the method comprises administering to the subject a first fluid composition that comprises the one or more reactive multi-arm polymers and a second fluid composition that comprises the one or more reactive multifunctional compounds. In some of these embodiments, the first fluid composition and the second fluid composition are delivered using a double barrel syringe.

In some embodiments, the method comprises administering to the subject a first fluid composition that comprises the one or more reactive multi-arm polymers and the one or more reactive multifunctional compounds and a second fluid composition that comprises an accelerant that accelerates formation of covalent crosslinks. In some of these embodiments, the first fluid composition and the second fluid composition are delivered using a double barrel syringe.

In other aspects, the present disclosure pertains to dye-containing hydrogel compositions that comprise a crosslinked reaction product of the one or more reactive multi-arm polymers, the one or more reactive multifunctional compounds, and the covalently attached dye molecule of any of the above aspects and embodiments, wherein the covalently attached dye molecule is covalently attached to the crosslinked reaction product.

In some embodiments, dye-containing hydrogel is in the form of injectable particles.

In some embodiments, which can be used in conjunction with the above aspects and embodiments, the dye-containing hydrogel comprises hydrolysable linkages. In some of these embodiments, the dye-containing hydrogel bioerodes in vivo over a period ranging anywhere from 1 day to 3 days to 1 week to 2 weeks to 1 month to 3 months to 6 months to 1 year to 2 years or longer (i.e., over a period ranging between any two of the preceding values).

In other aspects, the present disclosure pertains to methods of treatment comprising administering to a subject the dye-containing hydrogel composition of any the above aspects and embodiments.

Benefits of the hydrogels of the present disclosure include one or more of the following, among others: hydrogels are provided which are colored in the visible region of the electromagnetic spectrum, hydrogels are provided which are useful for various applications in which it is desirable for a health care professional to visibly locate hydrogels, the ability to dye hydrogels with different color for better physical consistency, the ability to provide colored hydrogels that are biodegradable, enabling complete removal of the hydrogel even when small amounts are left behind after a medical procedures.

The above and other aspects, embodiments, features and benefits of the present disclosure will be readily apparent from the following detailed description.

### BRIEF DESCRIPTION OF THE DRAWINGS

FIGS. 1A-1D schematically illustrate methods of forming reactive polymers, in accordance with four embodiments of the present disclosure.

FIGS. 2A-2F schematically illustrate methods of covalently attaching dye molecules, in accordance with six embodiments of the present disclosure.

FIG. 3 schematically illustrate a method of forming a primary amine group from a hydroxyl group, in accordance with an embodiment of the present disclosure.

FIG. 4 schematically illustrate a method of covalently attaching a dye molecule to a reactive polymer, in accordance with an embodiment of the present disclosure.

FIG. 5 schematically illustrate a method of covalently attaching a dye molecule to a reactive multifunctional compound, in accordance with an embodiment of the present disclosure.

FIGS. 6A-6C schematically illustrate methods of forming covalent linkages, in accordance with three embodiments of the present disclosure.

FIG. 7 illustrates a delivery device, in accordance with an embodiment of the present disclosure.

FIG. 8 illustrates a delivery device, in accordance with another embodiment of the present disclosure.

### DETAILED DESCRIPTION

In various embodiments, the present disclosure provides systems for forming hydrogels having a variety of colors.

The systems comprises (a) one or more reactive polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule. The dye molecule may be covalently linked to a reactive polymer, a reactive multifunctional compound, or both.

Covalently attached dye molecules in accordance with the present disclosure may have a color in the visible spectrum, for example, as a result of preferential reflection of incident visible light or as a result of absorption of incident radiation having first wavelength (e.g., ultraviolet or visible light) and re-emission of light having a longer wavelength in the visible spectrum (i.e., as a result of fluorescence). In some embodiments, a first dye molecule and a second dye molecule that is different from the first dye molecule may be covalently attached. For example, the first dye molecule may reflect light in the visible spectrum of a first wavelength and the second dye molecule may fluoresce light in the visible spectrum at a second wavelength after being exposed to radiation having a wavelength that is shorter than the second wavelength, among other possibilities.

In various embodiments, the system is configured to deliver the reactive polymer, the reactive multifunctional compound, and the covalently attached dye molecule under conditions such that covalent crosslinks are formed between the first reactive moieties of the reactive polymer and the second reactive moieties of the reactive multifunctional compound, thereby forming hydrogel, which contains a covalently attached dye molecule.

Particular examples of first and second reactive moieties include the following among others (a) first reactive moieties that comprise electrophilic groups and second reactive moieties that comprise nucleophilic groups, or vice versa, (b) first reactive moieties that comprise strained alkyne groups and second reactive moieties that comprise azide groups, or vice versa, and (c) first reactive moieties that comprise strained alkene groups and second reactive moieties that comprise tetrazine groups, or vice versa.

In some embodiments, reactive polymers in accordance with the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a first reactive moiety is covalently attached to an opposite end of the hydrophilic polymer segment through a suitable linkage, which linkage may further comprise a hydrolysable ester group in some instances. In some embodiments, a dye molecule is covalently attached to a fraction of the hydrophilic polymer segments through a suitable linkage, which may further comprise a hydrolysable ester group in some cases.

Reactive polymers in accordance with the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

First reactive moieties R₁ include moieties that comprise electrophilic groups, moieties that comprise nucleophilic groups, moieties that comprise strained alkyne groups, moieties that comprise strained alkene groups, moieties that comprise azide groups,

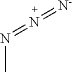

and moieties that comprise tetrazine groups

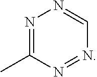

Electrophilic groups may be selected, for example, from cyclic imide ester groups, such as succinimide ester groups,

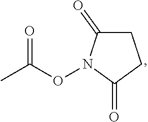

maleimide ester groups, glutarimide ester groups, diglycolimide ester groups, phthalimide ester groups, and bicyclo[2.2.1]hept-5-ene-2,3-dicarboxylic acid imide ester groups,

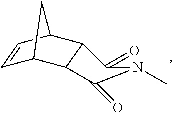

imidazole ester groups, imidazole carboxylate groups and benzotriazole ester groups, among other possibilities. Nucleophilic groups may be selected, for example, from amine groups, thiol groups, and hydroxyl groups, among other possibilities. Strained alkyne groups may be selected, for example, from (1R,8S,9s)-bicyclo[6.1.0]non-4-yn-9-yl groups,

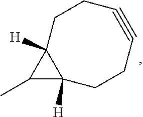

and dibenzocyclooctyne groups, among other possibilities. Strained alkene groups may be selected, for example, from cyclooct-4-en-1-yl groups,

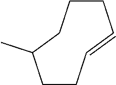

Cyclooct-4-enyl 2,5-dioxopyrrolidin-1-yl carbonate, Carbamic acid, N-(3-aminopropyl)-, 4-cycloocten-1-yl ester, Carbamic acid, N-[2-[2-(2-aminoethoxy) ethoxy]ethyl]-, 4-cycloocten-1-yl ester groups, among other possibilities.

The electrophilic groups, nucleophilic groups, strained alkyne groups, strained alkene groups, azide groups, or tetrazine groups may be linked to the hydrophilic polymer segment through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, or a linking moiety that comprises a combination of two or more of any of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising three or more hydroxyl groups, also referred to herein as a “polyol”, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Polyols having a biodegradable ester group may also be made from polyols such those described above. For example, a precursor polyol such as those described above may be reacted in a ring-opening reaction with a lactone (e.g., α-acetolactone, β-propiolactone, γ-butyrolactone, δ-valerolactone, ε-caprolactone, etc.) to form a further polyol that comprises a hydroxyl end group that is linked to a residue of precursor polyol through an alkyl group (e.g., a C₁-C₁₀ alkyl group) and a hydrolysable ester group at the site of each of the hydroxyl groups of polyol.

Iodinated polyols are desirable where radiopacity (e.g., visibility under fluoroscopy) is desired. Iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, and combinations thereof, among others. Specific examples of iodinated polyols include commercially available 1,3,5-triiodo-2,4,6-trishydroxymethylbenzene, iodixanol, iotrolan, iohexol, ioversol, iopamidol, iohexol impurity J, and iopromide, among others.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

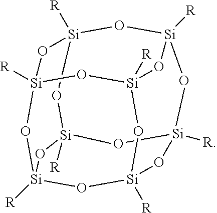

In the present disclosure, the R groups comprise the polymer arms described herein.

Reactive multi-arm polymers in accordance with the present disclosure can be formed from hydroxy-terminated multi-arm polymers having arms that comprise one or more hydroxyl end groups. In some embodiments of the present disclosure, a polyol such as one of those described below, among others, may be used as multifunctional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, U.S.A.

Although hydroxy-terminated multi-arm PEO polymers are used in the specific synthetic strategies described hereinbelow, it will be recognized that these strategies are widely applicable to hydroxy-terminated polymers having hydrophilic polymer segments other that PEO segments, such as the segments disclosed above.

In some embodiments, a hydroxy-terminated multi-arm hydrophilic polymer may be reacted with a cyclic anhydride to form carboxylic-acid-terminated polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

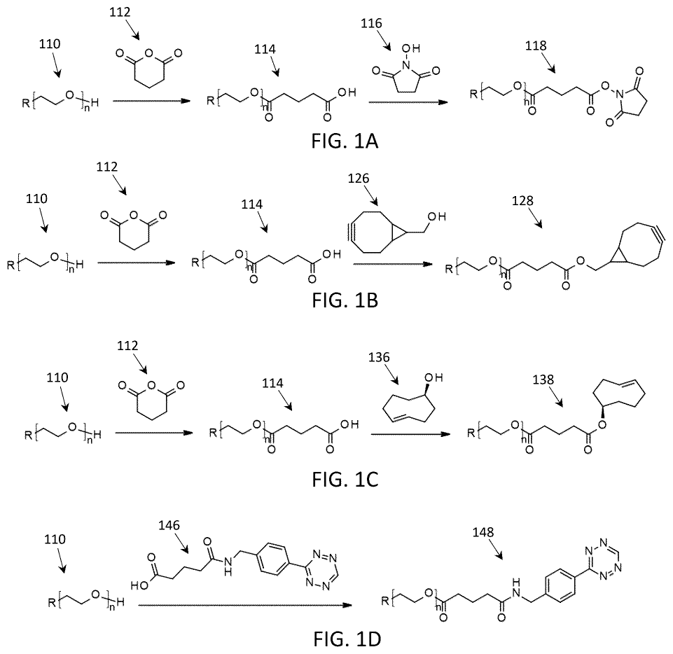

With reference now to FIG. 1A, a cyclic anhydride, specifically glutaric anhydride 112, is reacted with a hydroxy-terminated multi-arm hydrophilic polymer, specifically a hydroxy-terminated multi-arm PEO 110, where R corresponds to a core, to form a carboxylic-acid-terminated multi-arm polymer, specifically a glutaric-acid-terminated multi-arm PEO 114. (Although only one polymer arm of the multi-arm polyethylene oxide 110 is shown attached to the core R in FIGS. 1A-D, it is to be understood that additional polymer arms are present.) In FIGS. 1A-1D, n is an integer and may have a value ranging from 5 to 1000 or more. Although specific example using multi-arm PEO are illustrated, the preceding strategy is widely applicable to hydroxy-terminated polymers having hydrophilic polymer segments other PEO segments, such as the segments disclosed above.

A first reactive moiety may then be linked to the carboxylic-acid-terminated polymer. In some embodiments, an electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In a particular embodiment shown in FIG. 1A, a carboxylic-acid-terminated polymer, specifically, glutaric-acid-terminated multi-arm PEO 114, is reacted with N-hydroxysuccinimide 116 in the presence of a suitable amide coupling agent, such as a carbodiimide coupling agent, to form a succinimidyl-glutarate-terminated multi-arm PEO 118. Although a specific example using multi-arm PEO is illustrated, the preceding strategy is widely applicable to multi-arm polymers having hydrophilic polymer segments other PEO segments, such as the segments disclosed above. Succinimidyl-glutarate-terminated multi-arm polymers are also available commercially. For example, succinimidyl-glutarate-terminated four-arm PEG and succinimidyl-glutarate-terminated eight-arm PEG are available from JenKem Technology USA.

In some embodiment, the present disclosure provides a mixture of reactive multi-arm polymers, wherein a first fraction of the reactive multi-arm polymers comprise a covalently attached dye molecule, and wherein a second fraction of the reactive multi-arm polymers in the mixture do not comprise the covalently attached dye molecule. For example, the first fraction of the reactive multi-arm polymers comprising the covalently attached dye molecule may range, for example, from 0.00001 mol % or less to 0.1 mol % or more of the reactive multi-arm polymers (e.g., ranging anywhere from 0.00001 mol % to 0.0001 mol % to 0.001 mol % to 0.01 mol % to 0.1 mol %) and the second fraction of the reactive multi-arm polymers that do not comprise a covalently attached dye molecule may range, for example, from 99.9 mol % or less to 99.99999 mol % or more of the reactive multi-arm polymers (e.g., ranging anywhere from 99.9 mol % to 99.99 mol % to 99.999 mol % to 99.9999 mol % to 99.99999 mol %). In some these embodiments, the weight of the reactive multi-arm polymers that comprise the covalently attached dye molecule in the mixture may range from 0.1 ppm or less to 1000 ppm or more (e.g., ranging anywhere from 0.1 ppm to 1.0 ppm to 10 ppm to 100 ppm to 1000 ppm) of the total weight of the reactive multi-arm polymers in the mixture. The particular first fraction selected will depend in some cases on the color intensity of the covalently attached dye molecule. The dye molecule is covalently attached to the hydrophilic polymer segment through a suitable linkage, which may further comprise a hydrolysable ester group in some instances.

In some embodiments, the present disclosure provides processes wherein dye molecules are covalently attached to hydrophilic polymer segments of carboxylic-acid-terminated polymers.

In some embodiments, dye molecules are covalently attached to hydrophilic polymer segments through linkages that comprise an amide group.

For example, polymer arms having cyclic-imide-containing end groups such as those described above may be reacted with primary-amine-containing dye molecules under basic conditions to form covalent linkages that contain an amide group between the dye molecule and one portion of the polymer arms in the reaction mixture, while another portion of the polymer arms having cyclic-imide-containing end groups in the reaction mixture remain unchanged.

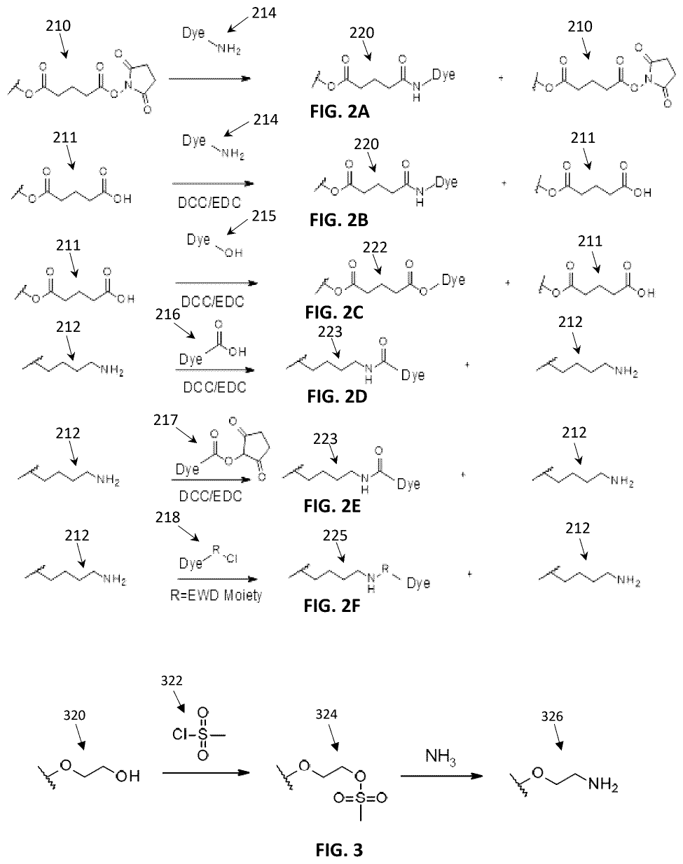

For example, with reference to FIG. 2A, a portion of the succinimidyl-glutarate-terminated arms 210 of a succinimidyl-glutarate-terminated multi-arm polymer in a reaction mixture may be reacted with a primary-amine-containing dye molecule 214 under basic conditions to form a mixture of multi-arm polymers in which a portion of the polymer arms in the product mixture are dye-terminated arms 220 and in which another portion of the polymer arms in the product mixture remain unchanged/unreacted as succinimidyl-glutarate-terminated arms 210.

In other embodiments, polymer arms having carboxylic-acid-terminated arms such as those described above may be reacted with primary-amine-containing dye molecules in the presence of a suitable coupling agent to form a covalent linkage that contains an amide group between the dye molecule and a portion of the polymer arms in the reaction mixture, while another portion of the polymer arms having carboxylic acid end groups remain unchanged/unreacted.

For example, as shown in FIG. 2B, a portion of the carboxylic-acid-terminated arms 211 of a glutaric-acid-terminated multi-arm polymer may be reacted in a reaction mixture with a primary-amine-containing dye molecule 214 in the presence of a suitable coupling agent such as DCC or EDC to form a mixture of multi-arm polymers in which a portion of the polymer arms in the product mixture are dye-terminated arms 220 and in which another portion of the polymer arms in the product mixture remain unreacted as glutaric-acid-terminated arms 211.

Then, in some embodiments, the portion of the polymer arms that have carboxylic-acid-terminated groups may be reacted with an N-hydroxy cyclic imide compound in the presence of a suitable coupling agent as previously described to form a mixture of multi-arm polymers, in which a portion of the polymer arms in the product mixture are dye-terminated arms and in which another portion of the polymer arms in the product mixture are N-hydroxy-cyclic-imide-terminated arms.

For example, glutaric-acid-terminated arms 211 in the product mixture of the process of FIG. 2B may be reacted with N-hydroxysuccinimide in the presence of a coupling agent (e.g., along the lines described in conjunction with FIG. 1A) to form succinimidyl-glutarate-terminated polymer arms 210 like that of FIG. 2A. One portion of the polymer arms in the resulting product mixture remain unreacted as dye-terminated arms 220 and another portion of the polymer arms in the product mixture are newly formed succinimidyl-glutarate-terminated arms 210.

In some embodiments, a dye molecule is covalently attached to a hydrophilic polymer segment of a polymer arm through a linkage that comprises an ester group.

For instance, a portion of the carboxylic-acid-terminated arms of a carboxylic-acid-terminated multi-arm polymer may be reacted in a reaction mixture with a hydroxyl-group-containing dye molecule in the presence of a suitable coupling agent such as DCC or EDC to form a mixture of multi-arm polymers in which a portion of the polymer arms in the resulting product mixture are dye-molecule-terminated arms and in which another portion of the polymer arms in the product mixture remain unreacted as carboxylic-acid-terminated arms.

For example, with reference to FIG. 2C, a portion of the glutaric-acid-terminated arms 211 of a glutaric-acid-terminated multi-arm polymer in a reaction mixture may be reacted with a hydroxyl-containing dye molecule 215 under in the presence of DCC or EDC to form product mixture in which a portion of the polymer arms in the product mixture are dye-molecule-terminated arms 222 and in which another portion of the polymer arms in the product mixture remain unreacted as glutaric-acid-terminated arms 211.

Then, in some embodiments, the portion of the polymer arms that have carboxylic-acid-terminated groups may be reacted with an N-hydroxy cyclic imide compound in the presence of a suitable coupling agent as previously described to form a mixture of multi-arm polymers, in which a portion of the polymer arms in the product mixture are dye-terminated arms and in which another portion of the polymer arms in the product mixture are N-hydroxy-cyclic-imide-terminated arms.

For example, the glutaric-acid-terminated arms 211 in the product mixture of the process of FIG. 2C may be reacted with N-hydroxysuccinimide in the presence of a coupling agent (e.g., along the lines described in conjunction with FIG. 1A) to form succinimidyl-glutarate-terminated polymer arms 210 like that of FIG. 2A. A portion of the polymer arms the resulting product mixture remain unreacted as dye-terminated arms 222 and another portion of the polymer arms in the product mixture are newly formed succinimidyl-glutarate-terminated arms 210.

In some embodiments, dye molecules are covalently attached to primary-amine-terminated polymers.

Primary-amine-terminated polymers may be formed from hydroxyl-terminated polymers. In a particular example shown in FIG. 3, a hydroxy-terminated polymer, for example, a multi-arm polymer having arms that comprise one or more hydroxyl end groups, such as a hydroxy-terminated multi-arm polymer 320 (only a single hydroxyl end group of a single arm is illustrated; the remainder of the hydroxy-terminated polymer is not illustrated) is first treated with methanesulfonyl chloride to form an intermediate methanesulfonate-terminated multi-arm polymer 324 in which hydroxyl groups of the hydroxy-terminated polymer are converted into methanesulfonate groups (only a single methanesulfonate end group of a single arm is illustrated; the remainder of the methanesulfonate-terminated polymer is not illustrated). The methanesulfonate groups are then reacted with ammonia to form an amino-terminated multi-arm polymer 326 in which methanesulfonate groups of the methanesulfonate-terminated multi-arm polymer 324 are converted into amino groups (only a single amino end group of a single arm is illustrated; the remainder of the amino-terminated polymer is not illustrated).

In some embodiments, primary amine groups may be linked to a carboxylic-acid-terminated polymer to form a primary-amine-terminated polymer. For example, a diamine compound such as 1,2-diaminoethane, 1,3-diaminoproane, 1,4-diaminobutane, etc., in which one of the amino groups of the diamine compound is protected with a suitable protective group (e.g., a tert-butyloxycarbonyl (tBoc) or a fluorenylmethyloxycarbonyl (Fmoc) or a benzyloxycarbonyl (Cbz) protective group) can be reacted in an amide coupling reaction with carboxylic acid groups of a carboxylic-acid-terminated polymer such as those described above (e.g., a glutaric-acid-terminated multi-arm PEO 114 illustrated in FIG. 1A) in the presence of a suitable coupling agent, such as a carbodiimide coupling agent, to form an amino-terminated polymer, for example, an amino-C₁-C₄-alky-terminated polymer in which the amino-C₁-C₄-alkyl groups are linked to the hydrophilic polymer segments through an amide group and a hydrolysable ester group.

In other embodiments, the partially protected diamine compound can be reacted in an amide coupling reaction with carboxylic acid groups of a carboxylic-acid-terminated polymer that does not contain a hydrolysable ester group to form an amino-terminated polymer, for example, an amino-C₁-C₄-alkyl-terminated polymer in which the amino-C₁-C₄-alkyl groups are linked to the hydrophilic polymer segments through an amide group, but not through a hydrolysable ester group.

Primary-amine-terminated multi-arm polymers are also commercially available. For example, primary-amine-terminated four-arm PEG, primary-amine-terminated six-arm PEG and primary-amine-terminated eight-arm PEG are available from JenKem Technology USA.

In some embodiments, a portion of the primary-amine-terminated arms of a primary-amine-terminated multi-arm polymer may be reacted in a reaction mixture with a carboxylic-acid-group-containing dye molecule in the presence of a suitable coupling agent such as DCC or EDC to form a mixture of multi-arm polymers in which a portion of the polymer arms in the product mixture are dye-molecule-terminated arms in which the dye molecule is covalently attached to the multi-arm polymer through a linkage that comprises an amide group and in which another portion of the polymer arms in the product mixture remain unreacted as primary-amine-terminated arms.

For example, with reference to FIG. 2D, a portion of the primary-amine-terminated arms 212 of a primary-amine-terminated multi-arm polymer in a reaction mixture may be reacted with a carboxylic-acid-containing dye molecule 216 in the presence of DCC or EDC to form a mixture of multi-arm polymers wherein a portion of the polymer arms in the resulting product mixture are dye-molecule-terminated polymer arms 223 and in which another portion of the polymer arms in the product mixture remain unreacted as primary-amine-terminated polymer arms 212.

In some embodiments, a portion of the primary-amine-terminated arms of a primary-amine-terminated multi-arm polymer may be reacted in a reaction mixture with a cyclic-imide-ester-containing dye molecule to form a mixture of multi-arm polymers in which a portion of the polymer arms in the reaction mixture are dye-molecule-terminated arms in which the dye molecule is covalently attached to the multi-arm polymer through a linkage that comprises an amide group and in which another portion of the polymer arms in the reaction product remain unreacted as primary-amine-terminated arms.

For example, with reference to FIG. 2E, a portion of the primary-amine-terminated arms 212 of a primary-amine-terminated multi-arm polymer in a reaction mixture may be reacted with a cyclic-imide-ester-containing dye molecule 217 to form a mixture of multi-arm polymers wherein a portion of the polymer arms in the product mixture are dye-molecule-terminated polymer arms 223 and in which another portion of the polymer arms in the product mixture remain unreacted as primary-amine-terminated polymer arms 212.

In some embodiments, reactive dyes may be employed, including dyes comprising one or more of the following reactive groups: halo-triazine groups such as monochloro-triazine groups, monofluoro-monochloro-triazine groups, dichloro-triazine groups, monochloro-monohydroxy-triazine groups, monochloro-monoamino-triazine groups, and monofluoro-monoamino-triazine groups, halo-pyrimidine groups, such as difluoro-chloro-pyrimidine groups, trichloro-pyrimidine groups, and fluoro-chloro-pyrimidine groups, halo-quinoxaline groups such as dichloro-quinoxaline groups, vinyl sulfone groups. Such reactive dyes may then be reacted with hydrophilic polymers having suitable nucleophilic groups such as amine groups or thiol groups to form hydrophilic polymers with covalently attached dyes.

For example, in some embodiments, a portion of the primary-amine-terminated arms of a primary-amine-terminated multi-arm polymer may be reacted in a reaction mixture with a reactive dye molecule to form a mixture of multi-arm polymers in which a portion of the polymer arms in the reaction mixture are dye-molecule-terminated arms in which the dye molecule is covalently attached to the polymer arm and in which another portion of the polymer arms in the reaction product remain unreacted as primary-amine-terminated arms.

For example, with reference to FIG. 2F, a portion of the primary-amine-terminated arms 212 of a primary-amine-terminated multi-arm polymer in a reaction mixture may be reacted with a halo-substituted reactive dye 218, where R represents an electron withdrawing (EWD) moiety such as triazine-containing moiety to form a mixture of multi-arm polymers wherein a portion of the polymer arms in the product mixture are dye-molecule-terminated polymer arms 225 and in which another portion of the polymer arms in the product mixture remain unreacted as primary-amine-terminated polymer arms 212.

In some embodiments, reactive dye molecules may be converted to further dye molecules, which can then be reacted with a suitable multi-arm polymer to form a mixture of multi-arm polymers wherein a portion of the polymer arms in the product mixture are dye-molecule-terminated polymer arms and in which another portion of the polymer arms in the product mixture remain unreacted.

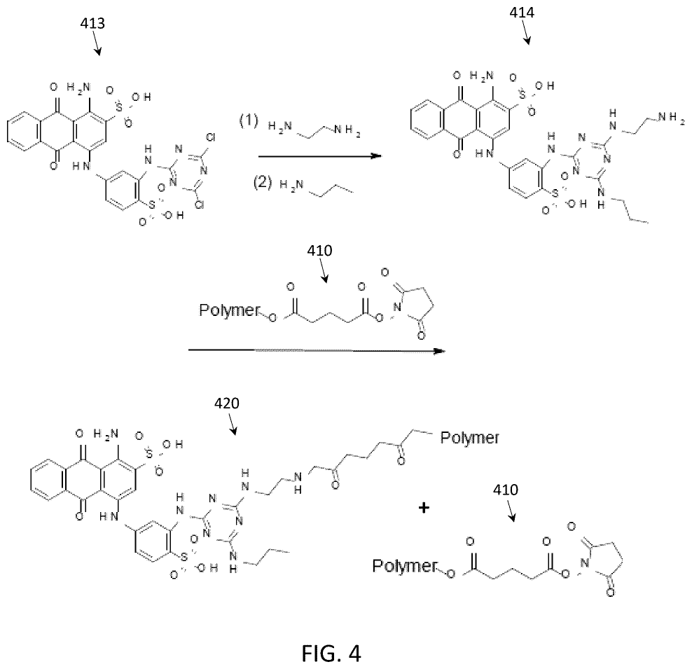

For example, with reference to FIG. 4, in a first step, an excess of a dye comprising one or more halo-triazine groups, specifically, reactive blue 4 413 is reacted with a diamine, specifically, a C₁-C₄ diamine such as ethylenediamine, followed by an reaction with a C₁-C₄ alkyl monoamine, specifically ethylamine, to provide a monoamine-substituted dye molecule, specifically, monoamine-functionalized reactive blue 4 414. The amine-functionalized reactive blue 4 414 is then reacted in a reaction mixture with a cyclic-amide-terminated multi-arm polymer, specifically, multi-arm polymer that contains succinimidyl-glutarate-terminated PEO arms 410, under basic conditions to form a mixture of multi-arm polymers in which a portion of the polymer arms in the product mixture are dye-terminated arms, specifically, dye-terminated PEO arms 420 and in which another portion of the polymer arms in the product mixture remain unchanged/unreacted as succinimidyl-glutarate-terminated PEO arms 410. By tuning the amount of reactive blue 4 in the synthesis, only a small fraction of the star polymer arms will be consumed, while providing the necessary color.

As previously noted, in various embodiments, reactive multi-arm polymers in accordance with the present disclosure can be formed from carboxylic-acid-terminated multi-arm polymers having arms that comprise one or more carboxylic acid end groups. In some of these embodiments, the one or more carboxylic acid end groups are linked to hydrophilic polymer segments through hydrolysable ester groups. Examples of such polymers include cyclic-imide-ester-terminated multi-arm polymers such as the succinimidyl-glutarate-terminated multi-arm PEO 118 of FIG. 1A.

In further embodiments, strained alkyne groups may be linked to a carboxylic-acid-terminated polymer. For example, in a particular embodiment shown in FIG. 1B, a glutaric-acid-terminated multi-arm PEO 114 is reacted with a hydroxyl-substituted strained alkyne such as (1R,8S,9s)-bicyclo[6.1.0]non-4-yn-9-ylmethanol 126 in an ester coupling reaction in the presence of a suitable coupling agent to produce (1R,8S,9s)-bicyclo[6.1.0]non-4-yn-9-yl-glutarate-terminated multi-arm PEO 128, in which (1R,8S,9s)-bicyclo[6.1.0]non-4-yn-9-yl groups are coupled to the polymer arms through two hydrolysable ester groups. In an alternative embodiment (not shown), a carboxylic-acid-terminated multi-arm PEO is reacted with an amine-substituted strained alkyne such as (1R,8S,9s)-bicyclo[6.1.0]non-4-yn-9-ylmethamine in an amide coupling reaction in the presence of a suitable coupling agent to produce a multi-arm PEO in which (1R,8S,9s)-bicyclo[6.1.0]non-4-yn-9-yl groups are coupled to the polymer arms through an amide group and a hydrolysable ester group.

In some embodiments, only a portion of the carboxylic-acid-terminated arms of a carboxylic-acid-terminated multi-arm polymer may be reacted, thereby forming a mixture of multi-arm polymers in which a portion of the polymer arms in the product mixture are strained-alkyne-terminated arms and in which another portion of the polymer arms in the product mixture remain unreacted as carboxylic-acid-terminated arms. Such polymers may be formed by selecting a suitable molar ratio between the carboxylic-acid-terminated polymer and either the hydroxyl-substituted strained alkyne or the amine-substituted alkyne.

In some embodiments, the portion corresponding to the carboxylic-acid-terminated arms can then be reacted with a primary-amine-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an amide group (or an amide group and an ester group in some embodiments as seen, for example, in the dye-terminated arms 220 of FIG. 2B) and in which another portion of the polymer arms in the reaction product remain unreacted as strained-alkyne-terminated arms.

In some embodiments, the portion corresponding to the carboxylic-acid-terminated arms can be reacted with a hydroxyl-group-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an ester group (or two ester groups in some embodiments as seen, for example, in the dye-terminated arms 222 of FIG. 2C) and in which another portion of the polymer arms in the reaction product remain unreacted as strained-alkyne-terminated arms.

In some embodiments, strained alkene groups may be linked to the carboxylic-acid-terminated polymer. For example, in a particular embodiment shown in FIG. 1C, glutaric-acid-terminated multi-arm PEO 114 is reacted with a hydroxyl-substituted strained alkene such as cyclooct-4-en-1-ol 136 in an ester coupling reaction in the presence of a suitable coupling agent to produce cyclooct-4-en-1-yl-glutarate-terminated multi-arm PEO 138, in which cyclooct-4-en-1-yl groups are coupled to the polymer arms through two hydrolysable ester groups. In an alternative embodiment (not shown), carboxylic-acid-terminated multi-arm PEO is reacted with an amine-substituted strained alkene such as cyclooct-4-en-1-amine in an amide coupling reaction in the presence of a suitable coupling agent to produce a multi-arm PEO in which cyclooct-4-en-1-yl groups are coupled to the polymer arms through an amide group and a hydrolysable ester group.

In some embodiments, only a portion of the carboxylic-acid-terminated arms of the carboxylic-acid-terminated multi-arm polymer may be reacted, thereby forming a mixture of multi-arm polymers in which a portion of the polymer arms in the product mixture are strained-alkene-terminated arms and in which another portion of the polymer arms in the product mixture remain unreacted as carboxylic-acid-terminated arms.

In some embodiments, the portion corresponding to the carboxylic-acid-terminated arms can then be reacted with a primary-amine-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an amide group (or an amide group and an ester group in some embodiments as seen, for example, in the dye-terminated arms 220 of FIG. 2B) and in which another portion of the polymer arms in the reaction product remain unreacted as strained-alkene-terminated arms.

In some embodiments, the portion corresponding to the carboxylic-acid-terminated arms can be reacted with a hydroxyl-group-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an ester group (or two ester groups in some embodiments as seen, for example, in the dye-terminated arms 222 of FIG. 2C) and in which another portion of the polymer arms in the reaction product remain unreacted as strained-alkene-terminated arms.

In some embodiments, tetrazine groups may be linked to a hydroxyl-terminated polymer. Exemplary hydroxyl-terminated polymers are described above and include the hydroxy-terminated multi-arm polyethylene oxide (PEO) 110 of FIG. 1A, among many others. In embodiments where a hydrolysable ester linkage is desired as well as a hydroxyl end group, hydroxyl groups of a hydroxyl-terminated polymer, such as the hydroxy-terminated multi-arm polyethylene oxide (PEO) 110 of FIG. 1A, among many others, may be reacted in a ring-opening reaction with a lactone (e.g., α-acetolactone, β-propiolactone, γ-butyrolactone, δ-valerolactone, ε-caprolactone, etc.) to form a hydroxyl-terminated polymer that comprises a hydroxyl end group that is linked to a residue of the hydroxyl-terminated polymer through an alkyl group (e.g., C₁-C₁₀ alkyl group) and a hydrolysable ester group at the site of each of the hydroxyl groups of the hydroxyl-terminated polymer.

In one example, a tetrazine based acid may be coupled to a hydroxyl-terminated polymer in an ester coupling reaction in the presence of a suitable coupling agent. In a particular embodiment shown in FIG. 1D, a tetrazine-terminated polymer is prepared by coupling a hydroxy-terminated multi-arm polyethylene oxide (PEO) 110 with 5-[4-(1,2,4,5-tetrazin-3-yl)benzylamino]-5-oxopentanoic acid in the presence of a carbodiimide coupling agent to produce 5-[4-(1,2,4,5-tetrazin-3-yl)benzylamino]-5-oxopentanoate-terminated multi-arm PEO 138. It is noted that the reactive tetrazine groups are linked to the multi-arm polymer through a hydrolysable ester group.

In some embodiments, only a portion of the hydroxyl-terminated arms of the hydroxyl-terminated multi-arm polymer are reacted, resulting in a mixture of multi-arm polymers in which a portion of the polymer arms in the product mixture are tetrazine-terminated arms and in which another portion of the polymer arms in the product mixture remain unreacted as hydroxyl-terminated arms.

In some embodiments, the portion corresponding to the hydroxyl-terminated arms can then be reacted with a carboxylic-acid-group-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an ester group and in which another portion of the polymer arms in the reaction product remain unreacted as tetrazine-terminated arms.

In some embodiments, an azide-terminated polymer may be formed from a hydroxyl-terminated polymer. For example, an azide-terminated polymer may be synthesized by reacting a hydroxy-terminated multi-arm polymer with methanesulfonyl chloride to obtain a methanesulfonyl-terminated multi-arm polymer. Then, sodium azide (NaN₃) is reacted with the methanesulfonyl-terminated multi-arm polymer to replace the methanesulfonate groups with azide groups, forming an azide-terminated multi-arm polymer.

In some embodiments, only a portion of the hydroxyl-terminated arms of the hydroxyl-terminated multi-arm polymer are reacted, resulting in a mixture of multi-arm polymers in which a portion of the polymer arms in the product mixture are azide-terminated arms and in which another portion of the polymer arms in the product mixture remain unreacted as hydroxyl-terminated arms.

In some embodiments, the portion corresponding to the hydroxyl-terminated arms can then be reacted with a carboxylic-acid-group-containing dye molecule in the presence of a suitable coupling agent to form a mixture of multi-arm polymers in which a portion of the polymer arms in the reaction mixture are dye-terminated arms in which a dye molecule is covalently attached to a hydrophilic polymer segment of a polymer arm through a linkage that comprises an ester group and in which another portion of the polymer arms in the reaction product remain unreacted as azide-terminated arms.

As previously indicated, the above strategies are applicable to a wide variety of polymers having hydrophilic polymer segments, other than the PEO segments typically exemplified, such as the hydrophilic polymer segments disclosed above.

From the above, it will be apparent that a wide range of dye molecules may be used for the synthetic schemes described herein, including dye molecules that contain amine groups, dye molecules that contain hydroxyl groups, dye molecules that contain thiol groups, dye molecules that contain carboxylic acid groups, dye molecules that contain halide groups, and so forth. Particular examples of dyes are shown in the table to follow. Although blue dye molecules are predominantly described, the methods described herein are applicable to any colored dye molecules.

| CAS# | Dye Name | Structure |
|---|---|---|
| 314-13-6 | Evans blue (dye) | 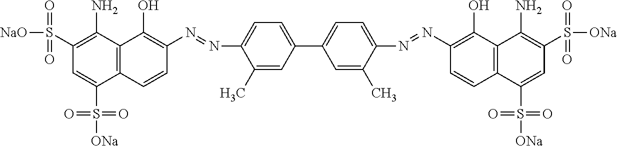 |
| 3521-06-0 | Basic Blue | 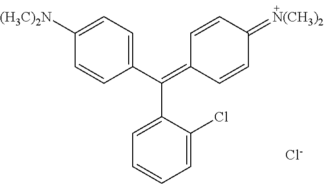 |
| 13324-20-4 | Reactive Blue 4 | 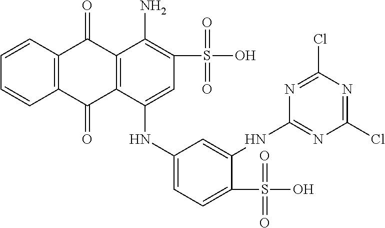 [SMILES: Nc1c(S(=O)(=O)O)cc(Nc2ccc(S(=O)(=O)O)c(Nc3nc(Cl)nc(Cl)n3)c2)c2c1C(=O)c1ccccc1C2=O] |
| 92-31-9 | Toluidine blue O, metachromatic dye | 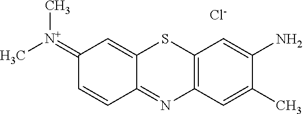 |
| 6424-85-7 | Acid blue 40 | 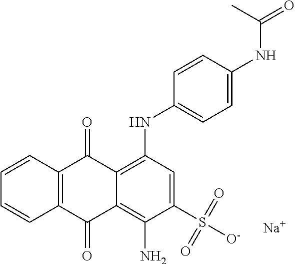 [SMILES: CC(=O)Nc1ccc(Nc2cc(S(=O)(=O)[O-])c(N)c3c2C(=O)c2ccccc2C3=O)cc1 ; [Na+]] |
| 72-57-1 | Trypan Blue | 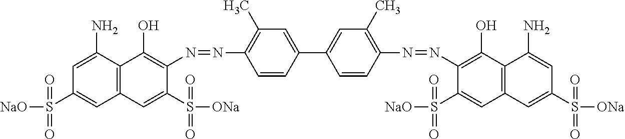 |
| 15791-78-3 | Disperse Blue 27 | 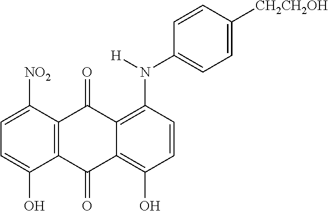 |
| 20298-86-6 | Phycocyanobilin | 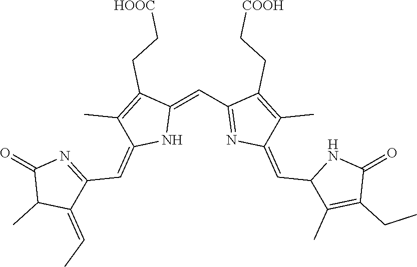 [SMILES: C/C=C1/C(/C=c2/[nH]/c(=C\C3=NC(=C/C4NC(=O)C(CC)=C4C)/C(C)=C3CCC(=O)O)c(CCC(=O)O)c2C)=NC(=O)C1C] |
| 84166-13-2 | Cibacron Blue 3G-A, Anthraquinone derivative dye | 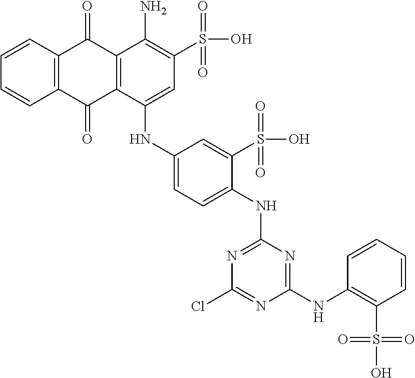 [SMILES: Nc1c(S(=O)(=O)O)cc(Nc2ccc(Nc3nc(Cl)nc(Nc4ccccc4S(=O)(=O)O)n3)c(S(=O)(=O)O)c2)c2c1C(=O)c1ccccc1C2=O] |
| 81-88-9 | Rhodamine B | 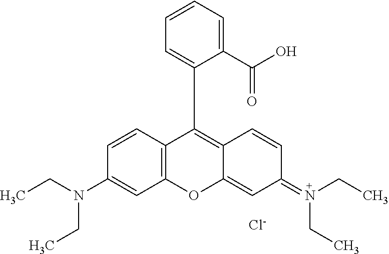 |
| 2580-78-1 | RB19 | 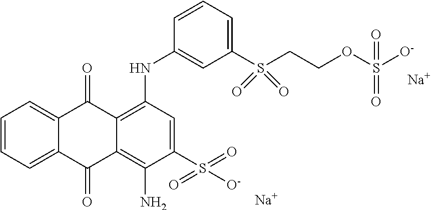 [SMILES: Nc1c(S(=O)(=O)[O-])cc(Nc2cccc(S(=O)(=O)CCOS(=O)(=O)[O-])c2)c2c1C(=O)c1ccccc1C2=O ; [Na+] ; [Na+]] |
| 6397-02-0 | Acid Blue 129 | 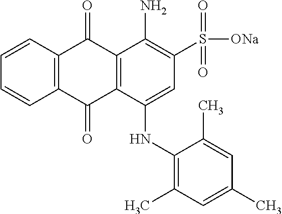 |
| 3179-90-6 | Disperse Blue 7 | 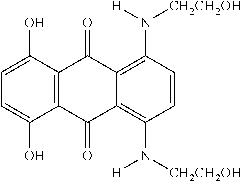 |
| 2375652-87-0 | 2′,5′-bis((4- aminophenyl) ethynyl)- [1,1′:4′,1″- terphenyl]- 4,4″- dicarbaldehyde | 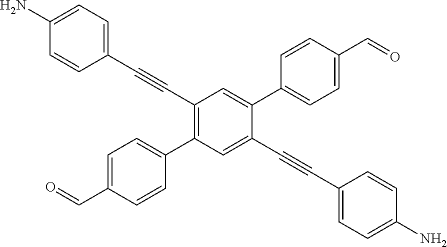 [SMILES: Nc1ccc(C#Cc2cc(-c3ccc(C=O)cc3)c(C#Cc3ccc(N)cc3)cc2-c2ccc(C=O)cc2)cc1] |
| 6359-98-4 | Acid yellow 17 | 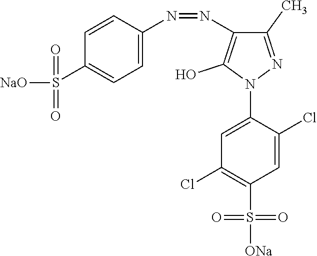 |
| 117548-22-8 | Amine-reactive green fluorescent dye | 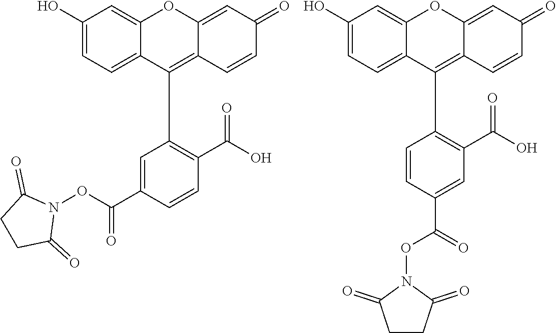 |

As previously indicated, in addition to a reactive polymer comprising a plurality of first reactive moieties as described above, which reactive polymer may or may not further include a covalently attached dye molecule, the systems described herein further comprise a reactive multifunctional compound comprising a plurality of second reactive moieties, wherein the first reactive moieties and the second reactive moieties are selected to covalently crosslink with one another to form a crosslinked reaction product, specifically, a hydrogel.

Various reactive multifunctional compounds that comprise a plurality of second reactive moieties that covalently react with the first reactive moieties of the reactive polymers will now be described.

As with the reactive polymers, the reactive multifunctional compounds may further comprise a covalently attached dye molecule in some embodiments. In this regard, multifunctional-compound-containing compositions in accordance with the present disclosure may be provided that contain a mixture of multifunctional compounds wherein a first fraction of the multifunctional compounds in the mixture comprise a covalently attached dye molecule and a second fraction of the multifunctional compounds in the mixture do not comprise a covalently attached dye molecule. For example, the first fraction of the multifunctional compounds comprising the covalently attached dye molecule may range, for example, from 0.00001 mol % or less to 0.1 mol % or more of the multifunctional compounds (e.g., ranging anywhere from 0.00001 mol % to 0.0001 mol % to 0.001 mol % to 0.01 mol % to 0.1 mol %) and the second fraction of the multifunctional compounds that do not comprise a covalently attached dye molecule may range, for example, from 99.9 mol % or less to 99.99999 mol % or more of the multifunctional compounds (e.g., ranging anywhere from 99.9 mol % to 99.99 mol % to 99.999 mol % to 99.9999 mol % to 99.99999 mol %). In some embodiments, the weight of multifunctional compound comprising the covalently attached dye molecule in the mixture may range from 0.1 ppm or less to 1000 ppm or more (e.g., ranging anywhere from 0.1 ppm to 1.0 ppm to 10 ppm to 100 ppm to 1000 ppm) of the total weight of multifunctional compound in the mixture.

In some embodiments, the multifunctional compounds are compounds having two or more nucleophilic groups, for example, the multifunctional compounds may have 2, 3, 4, 5, 6, 7, 8, 9, 10, 11, 12, 13, 14, 15, 16, 17, 18, 19, 20 or more nucleophilic groups. Specific examples of such multifunctional compounds include, for example, polyamines that contain at two or more amino (—NH₂) groups (e.g., 2, 3, 4, 5, 6, 7, 8, 9, 10 or more amino groups in some embodiments), also referred to herein as polyamino compounds. Polyamino compounds suitable for use in the present disclosure include polyamino compounds that comprise two or more primary amine groups, for example, —(CH₂)ₓ—NH₂ groups where x is 1, 2, 3, 4, 5 or 6. Polyamino compounds suitable for use in the present disclosure include polyamino compounds that comprise two or more basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further examples of polyamino compounds which may be used as the multifunctional compound include ethylenediamine, ethylenetriamine, diethylene triamine, hexamethylenetriiamine, di(heptamethylene) triamine, di(trimethylene) triamine, bis(hexamethylene) triamine, tris(2-aminoethyl)amine, tris(3-aminopropyl)amine, 1,3,5-tris-(2-aminoethyl)-[1,3,5]triazinane-2,4,6-trione, N,N,N′-tris(2-aminoethyl)ethylenediamine, triethylene tetramine, tripropylene tetramine, N,N′,N′-tetrakis(2-aminoethyl)-1,2-ethanediamine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Compounds having at least two amino groups may also be made from polyols such those described above. For example, hydroxyl groups of a polyol may be reacted in an ester coupling reaction in the presence of a suitable coupling agent with a carboxyl group of a suitable amino acid compound in which the amino group of the amino acid is protected with a suitable protective group (e.g., a tert-butyloxycarbonyl (tBoc) protective group). Examples of amino acids may be selected, for example, from beta amino acids such as 3-aminopropanoic acid (also known as beta-alanine), gamma amino acids such as 4-aminobutanoic acid (also known as gamma-aminobutyric acid, or GABA), delta amino acids such as 5-aminopentanoic acid, epsilon amino acids such as 6-aminohexanoic acid, etc. Deprotection of the protective groups results in a compound in which a primary amine group is linked to a residue of the polyol through a hydrolysable ester group at the site of each of the hydroxyl groups of polyol.

Moreover, the polyamino compounds further comprise a covalently attached dye molecule in some embodiments. In this regard, polyamino-compound-containing compositions in accordance with the present disclosure may be provided that contain a mixture of polyamino compounds wherein a portion of the polyamino compounds in the mixture comprise a covalently attached dye molecule and another portion of the polyamino compounds in the mixture do not comprise a covalently attached dye molecule.

Dye molecules may be attached to polyamino compounds by a variety of synthetic schemes. For example, analogous to the process described in FIG. 2D above, a molar excess of a polyamino compound may be reacted in a reaction mixture with a carboxylic-acid-group-containing dye molecule in the presence of a suitable coupling agent such as DCC or EDC, thereby forming a mixture of polyamino compounds in which a portion of the polyamino compounds in the product mixture comprise a dye molecule that is covalently attached to a remainder of the compound through a linkage that comprises an amide group and in which another portion of the polyamino compounds in the product mixture remain unreacted.

As another example, analogous to the process described in FIG. 2E above, a molar excess of a polyamino compound may be reacted in a reaction mixture with a cyclic-imide-ester-containing dye molecule dye molecule under basic conditions, thereby forming a mixture of polyamino compounds in which a portion of the polyamino compounds in the product mixture comprise a dye molecule that is covalently attached to a remainder of the compound through a linkage that comprises an amide group and in which another portion of the polyamino compounds in the product mixture remain unreacted.

As another example, analogous to the process described in FIG. 2F above, a molar excess of a polyamino compound may be reacted in a reaction mixture with a reactive dye such as a halo-triazine substituted dye having a single halo-triazine group, thereby forming a mixture of polyamino compounds in which a portion of the polyamino compounds in the product mixture comprise a dye molecule that is covalently attached to a remainder of the compound through a linkage that comprises a triazine group and in which another portion of the polyamino compounds in the product mixture remain unreacted.

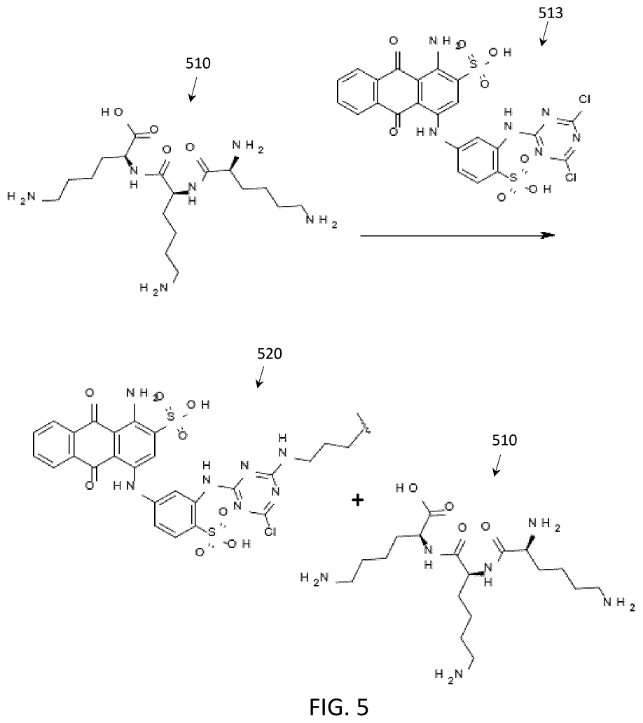

In a particular embodiment shown in FIG. 5, a molar excess of a polyamino compound, specifically, trilysine 510 is reacted in a reaction mixture with a reactive dye, specifically, reactive blue 4 513. The primary amine of one of the trilysines will substitute the chlorine in the reactive blue 4 to form a covalent bond. As a result, a mixture of polyamino compounds is formed in which a portion of the polyamino compounds in the product mixture are dye-containing molecules 520 that comprise a dye molecule that is covalently attached to a trilysine residue through a linkage that comprises a triazine group and in which another portion of the polyamino compounds in the product mixture correspond to unreacted trilysine molecules 510. By tuning the amount of reactive blue 4 in the synthesis, only a small fraction of the amine groups will be consumed, while providing the desired color.

In some embodiments, the multifunctional compound is a compound having two or more electrophilic groups (also referred to herein as polyelectrophilic compounds), for example, having 2, 3, 4, 5, 6, 7, 8, 9, 10, 11, 12, 13, 14, 15, 16, 17, 18, 19, 20 or more electrophilic groups. Electrophilic groups may be selected, for example, from cyclic imide ester groups, such as succinimide ester groups, maleimide ester groups, glutarimide ester groups, diglycolimide ester groups, phthalimide ester groups, and bicyclo[2.2.1]hept-5-ene-2,3-dicarboxylic acid imide ester groups, imidazole ester groups, imidazole carboxylate groups and benzotriazole ester groups, among other possibilities.

Commercially available compounds that contain at least two cyclic imide ester groups include disuccinimidyl glutarate (2 cyclic imide esters), 1,5-Bis(2,5-dioxo-1-pyrrolidinyl) 2-oxopentanedioate (2 cyclic imide esters), 1,5-Bis(2,5-dioxo-1-pyrrolidinyl) 3-hydroxypentanedioate (2 cyclic imide esters), and 1,2,3-Tris(2,5-dioxo-1-pyrrolidinyl) 2-hydroxy-1,2,3-propanetricarboxylate (3 cyclic imide esters). Compounds that contain at least two cyclic imide ester groups can also be formed from compounds having at least two carboxylic acid groups along the lines described in conjunction with FIG. 1A by reaction with an N-hydroxy cyclic imide compound, such as one of those described above, in the presence of a coupling agent. Compounds having at least two carboxylic acid groups also include various known polycarboxylic acid compounds such as glutaric acid, succinic acid, malonic acid, adipic acid, diglycolic acid, citric acid, isocitric acid, trimellitic acid, trimesic acid, aconitic acid, propane-1,2,3-tricarboxylic acid (tricarballylic acid), ethanetricarboxylic acid, and butane-1,2,3,4-tetracarboxylic acid, and ethane-1,1,2,2-tetracarboxylic acid, among others. In a specific example, a polycarboxylic acid compound is reacted with N-hydroxysuccinimide to form succinimidyl ester groups in the positions previously occupied by the carboxylic acid groups of the polycarboxylic acid compound.

Compounds that contain at least two cyclic imide ester groups can also be formed from polyols such those described above. For example, a polyol may be reacted with a cyclic anhydride compound described above in a ring opening reaction to form a polycarboxylic acid compound in which each of the carboxylic acid groups is linked to a residue of the polyol through a hydrolysable ester group. Then, the resulting polycarboxylic acid compound may be reacted with an N-hydroxy cyclic imide compound such as one of those described above in the presence of a coupling agent to form a compound in which cyclic imide ester groups are linked to a polyol residue through a ester linkage.

Dye molecules may also be attached to the polyelectrophilic compounds in some embodiments. For example, analogous to the process described in FIG. 2A above, a molar excess of a polyelectrophilic compound may be reacted in a reaction mixture with a primary-amine-containing dye molecule, thereby forming a mixture of polyelectrophilic compounds in which a portion of the polyelectrophilic compounds in the product mixture comprise a dye molecule that is covalently attached to a remainder of the compound through a linkage that comprises an amide group and in which another portion of the polyelectrophilic compounds in the product mixture remain unreacted.

In some embodiments, the multifunctional compound is a compound having at least two strained alkyne groups, for example, having 2, 3, 4, 5, 6, 7, 8, 9, 10, 11, 12, 13, 14, 15, 16, 17, 18, 19, 20 or more strained alkyne groups. Strained alkyne groups may be selected, for example, from (1R,8S,9s)-bicyclo[6.1.0]non-4-yn-9-yl groups and dibenzocyclooctyne groups, among other possibilities.

Commercially available compounds that contain at least two strained alkyne groups include 5,8-Dioxa-2,11-diazadodecanedioic acid, 1,12-bis[(1R,8S)-bicyclo[6.1.0]non-4-yn-9-ylmethyl] ester, Poly(oxy-1,2-ethanediyl) (CAS #1476737-97-9, α-[2-[[[(1α,8α,9β)-bicyclo[6.1.0]non-4-yn-9-ylmethoxy]carbonyl]amino]ethyl]-ω-[2-[[[(1α,8α,9β)-bicyclo[6.1.0]non-4-yn-9-ylmethoxy]carbonyl]amino]ethoxy] (CAS #2152700-22-4), 2-(9-bicyclo[6.1.0]non-4-ynylmethoxy)-˜{N}-[5-[[2-(9-bicyclo[6.1.0]non-4-ynylmethoxy)acetyl]amino]pentyl]acetamide (CAS #2743204-05-7), and 2-(9-bicyclo[6.1.0]non-4-ynylmethoxy)-˜{N}-[2-[2-[[2-(9-bicyclo[6.1.0]non-4-ynylmethoxy)acetyl]amino]ethoxy]ethyl]acetamide (CAS #2752158-76-0).

Compounds that contain at least two strained alkyne groups may also be formed from polycarboxylic acid compounds that contain at least two carboxylic acid groups.

Polycarboxylic acid compounds that contain at least two carboxylic acid groups include various known polycarboxylic acid compounds such as glutaric acid, succinic acid, malonic acid, adipic acid, diglycolic acid, citric acid, isocitric acid, trimellitic acid, trimesic acid, aconitic acid, propane-1,2,3-tricarboxylic acid (tricarballylic acid), ethanetricarboxylic acid, and butane-1,2,3,4-tetracarboxylic acid, and ethane-1,1,2,2-tetracarboxylic acid among others. Polycarboxylic acid compounds that contain at least two carboxylic acid groups may also be made from polyols as described above.

In one example, carboxylic acid groups of a polycarboxylic acid compound may be reacted with a hydroxyl-substituted strained alkyne such as (1R,8S,9s)-bicyclo[6.1.0]non-4-yn-9-ylmethanol in an ester coupling reaction in the presence of a suitable coupling agent to produce (1R,8S,9s)-bicyclo[6.1.0]non-4-yn-9-yl groups that are coupled to a residue of the polycarboxylic acid compound through two hydrolysable ester groups. In an alternative example, carboxyl groups of a polycarboxylic acid compound may be reacted with an amine-substituted strained alkyne such as (1R,8S,9s)-bicyclo[6.1.0]non-4-yn-9-ylmethamine in an amide coupling reaction in the presence of a suitable coupling agent to produce a compound in which (1R,8S,9s)-bicyclo[6.1.0]non-4-yn-9-yl groups are linked to a residue of the polycarboxylic acid compound through an amide group and a hydrolysable ester group.

Multifunctional compounds having at least two strained alkyne groups may also be provided with dye molecules.

For example, in some embodiments, analogous to the process of FIG. 2C, a molar excess of a polycarboxylic acid compound may be reacted in a reaction mixture with a hydroxyl-containing dye molecule in the presence of a suitable coupling agent such as DCC or EDC, thereby forming a mixture of polycarboxylic acid compounds in which a portion of the polycarboxylic acid compounds in the product mixture comprise a dye molecule that is covalently attached to a remainder of the compound through a linkage that comprises an ester group and in which another portion of the polycarboxylic acid compounds in the product mixture remain unreacted. In another example, analogous to the process of FIG. 2B, a molar excess of a polycarboxylic acid compound may be reacted in a reaction mixture with an amino-containing dye molecule in the presence of a suitable coupling agent such as DCC or EDC, thereby forming a mixture of polycarboxylic acid compounds in which a portion of the polycarboxylic acid compounds in the product mixture comprise a dye molecule that is covalently attached to a remainder of the compound through a linkage that comprises an amide group and in which another portion of the polycarboxylic acid compounds in the product mixture remain unreacted.

In either case, in a further step, a strained alkyne group can be attached to the remaining carboxylic acid groups along the lines described in conjunction with FIG. 1B, thereby forming a mixture of multifunctional compounds having a plurality of strained alkyne groups in which a portion of the multifunctional compounds in the product mixture comprise strained alkyne groups and a covalently attached dye molecule and in which another portion of the multifunctional compounds in the product mixture comprise strained alkyne groups but do not comprise a covalently attached dye molecule.

In some embodiments, the multifunctional compound is a compound having at least two strained alkene groups, for example, having 2, 3, 4, 5, 6, 7, 8, 9, 10, 11, 12, 13, 14, 15, 16, 17, 18, 19, 20 or more strained alkene groups. Strained alkene groups may be, for example, cyclooct-4-en-1-yl groups, among other possibilities.

Commercially available compounds that contain at least two strained alkene groups include 5,8,11,14,17,20,23,26-Octaoxa-2,29-diazatriacontanedioic acid, 1,30-di-4-cycloocten-1-yl ester, 5,8,11,14-Tetraoxa-2,17-diazaoctadecanedioic acid, 1,18-di-4-cycloocten-1-yl ester, Cyclooctene, 5,5′-[1,2-ethanediylbis(oxy-2,1-ethanediyloxy)]bis-, and Cyclooctene, 5,5′,5″-[(methylsilylidyne)tris(oxy)]tris-.

Compounds that contain at least two strained alkene groups may also be formed from polycarboxylic acid compounds that contain at least two carboxylic acid groups. For example, carboxylic acid groups of a polycarboxylic acid compound may be reacted with a hydroxyl-substituted strained alkene such as cyclooct-4-en-1-ol in an ester coupling reaction in the presence of a suitable coupling agent to produce a compound in which cyclooct-4-en-1-yl groups are linked to a residue of the polycarboxylic acid compound through two hydrolysable ester groups. In an alternative embodiment, carboxyl groups of a polycarboxylic acid compound may be reacted with an amine-substituted strained alkene such as cyclooct-4-en-1-amine in an amide coupling reaction in the presence of a suitable coupling agent to produce a compound in which cyclooct-4-en-1-yl groups are linked to a residue of the polycarboxylic acid compound through an amide group and a hydrolysable ester group.

Polycarboxylic acid compounds that contain at least two carboxylic acid groups include various known polycarboxylic acid compounds such as those described above. Polycarboxylic acid compounds that contain at least two carboxylic acid groups may also be made from polyols such as those described above.

Multifunctional compounds having at least two strained alkene groups may also be provided with dye molecules.

For example, in some embodiments, analogous to the process of FIG. 2C, a molar excess of a polycarboxylic acid compound may be reacted in a reaction mixture with a hydroxyl-containing dye molecule in the presence of a suitable coupling agent such as DCC or EDC, thereby forming a mixture of polycarboxylic acid compounds in which a portion of the polycarboxylic acid compounds in the product mixture comprise a dye molecule that is covalently attached to a remainder of the compound through a linkage that comprises an ester group and in which another portion of the polycarboxylic acid compounds in the product mixture remain unreacted. In another example, analogous to the process of FIG. 2B, a molar excess of a polycarboxylic acid compound may be reacted in a reaction mixture with an amino-containing dye molecule in the presence of a suitable coupling agent such as DCC or EDC, thereby forming a mixture of polycarboxylic acid compounds in which a portion of the polycarboxylic acid compounds in the product mixture comprise a dye molecule that is covalently attached to a remainder of the compound through a linkage that comprises an amide group and in which another portion of the polycarboxylic acid compounds in the product mixture remain unreacted.

In either case, in a further step, a strained alkene group can be attached to the remaining carboxylic acid groups along the lines described in conjunction with FIG. 1C, thereby forming a mixture of multifunctional compounds having a plurality of strained alkene groups in which a portion of the multifunctional compounds in the product mixture comprise strained alkene groups and a covalently attached dye molecule and in which another portion of the multifunctional compounds in the product mixture comprise strained alkene groups but do not comprise a covalently attached dye molecule.

In some embodiments, the multifunctional compound is a compound having at least two tetrazine groups, for example, having 2, 3, 4, 5, 6, 7, 8, 9, 10, 11, 12, 13, 14, 15, 16, 17, 18, 19, 20 or more tetrazine groups.

Commercially available compounds that contain at least two tetrazine groups include 5,5′-Bi-1,2,3,4-tetrazine.

Compounds that contain at least two tetrazine groups may also be formed from polyols that that contain at least two hydroxyl groups. Exemplary polyols include those described above for use in forming multi-arm polymers.

In some embodiments, a tetrazine based acid may be coupled to hydroxyl groups of a polyol that that contains at least two hydroxyl groups in an ester coupling reaction in the presence of a suitable coupling agent. In a particular example, multifunctional compound containing tetrazine groups may be prepared by coupling hydroxyl groups of a polyol with 5-[4-(1,2,4,trazin-3-yl)benzylamino]-5-oxopentanoic acid in the presence of a carbodiimide coupling agent to produce a compound in which 5-[4-(1,2,4,5-tetrazin-3-yl)benzylamino]-5-oxopentanoate groups are linked to a polyol residue at a site of each of the hydroxyl groups of the precursor polyol. It is noted that the 5-[4-(1,2,4,5-tetrazin-3-yl)benzylamino]-5-oxopentanoate groups contain a hydrolysable ester groups.

Multifunctional compounds having at least two tetrazine groups may also be provided with dye molecules.

For example, in some embodiments, a molar excess of a polyol may be reacted in a reaction mixture with a carboxylic-acid-containing dye molecule in the presence of a suitable coupling agent such as DCC or EDC, thereby forming a mixture of polyol compounds in which a portion of the polyol compounds in the product mixture comprise a dye molecule that is covalently attached to a remainder of the compound through a linkage that comprises an ester group and in which another portion of the polyol compounds in the product mixture remain unreacted. In a further step, a tetrazine based acid can be attached to the remaining hydroxyl groups in the product mixture along the lines described in conjunction with FIG. 1D, thereby forming a mixture of multifunctional compounds having a plurality of tetrazine groups in which a portion of the multifunctional compounds in the product mixture comprise tetrazine groups and a covalently attached dye molecule and in which another portion of the multifunctional compounds in the product mixture comprise tetrazine groups but do not comprise a covalently attached dye molecule.

In some embodiments, the multifunctional compound is a compound having at least two azide groups, for example, having 2, 3, 4, 5, 6, 7, 8, 9, 10, 11, 12, 13, 14, 15, 16, 17, 18, 19, 20 or more azide groups.

Commercially available compounds that contain at least two azide groups include 1,1′-Oxybis[2-(2-azidoethoxy)ethane], 1,17-Diazido-3,6,9,12,15-pentaoxaheptadecane, N,N′-(Dithiodi-2,1-ethanediyl)bis[4-azido-2-hydroxybenzamide], Propane, 1-azido-3-[(2-azidoethoxy) methoxy]-, 1,3,5-Tris(azidomethyl)-2,4,6-triethylbenzene.

Multifunctional compounds having at least two azide groups may be formed from polyols having at least two hydroxyl groups. Exemplary polyols include those described above for use in forming multi-arm polymers.

For example a multifunctional compound having at least two azide groups may be synthesized by reacting hydroxyl groups of a polyol with methanesulfonyl chloride to obtain multifunctional compound having at least two methanesulfonyl groups. Then, sodium azide (NaN₃) is reacted with the multifunctional compound having at least two methanesulfonyl groups to replace the methanesulfonate groups with azide groups, thereby forming a multifunctional compound having at least two azide groups.

Multifunctional compounds having at least two azide groups may also be provided with dye molecules. For example, in some embodiments, the reaction stoichiometry in the prior paragraph may be modified such that mixture of multifunctional compounds is formed in which a smaller portion of the multifunctional compounds in the product mixture comprise a hydroxyl group from the polyol that has not replaced by an azide group and in which a larger portion of the multifunctional compounds in the product mixture has had all of the hydroxyl groups replaced by azide groups. Then, a carboxylic-acid-containing dye molecule may be reacted with the remaining hydroxyl groups in the presence of a suitable coupling agent to link the dye through an ester-containing linkage.

As previously indicated, in some aspects, the present disclosure provides a dye-containing hydrogel that comprises a crosslinked reaction product of (a) a reactive polymer as described herein and (b) a reactive multifunctional compound as described herein, wherein a dye molecule is covalently attached to the crosslinked reaction product. In various embodiments, the reactive polymer will have three or more first reactive moieties and the multifunctional compound will have two or more second reactive moieties.

Three specific examples of covalent crosslinking reactions between first and second reactive moieties are shown in FIGS. 4A-4C.

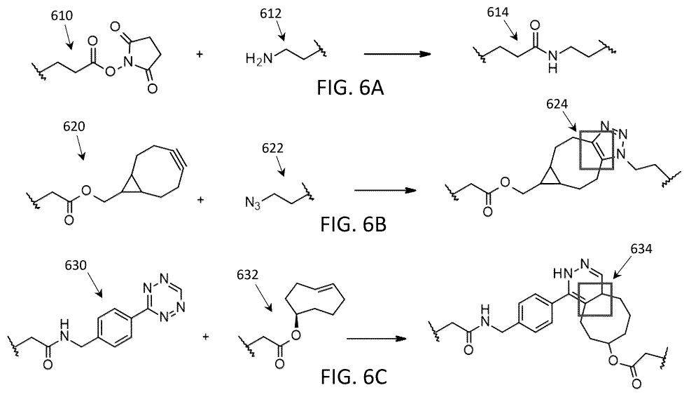

FIG. 6A shows a covalent crosslinking reaction between a cyclic amide ester group, specifically, a succinimide ester group 610 and a primary amine group 612, whereby an amide linking group 614 is formed. In this scheme, the first reactive moiety may comprise the cyclic amide ester group and the second reactive moiety may comprise the primary amine group, or the first reactive moiety may comprise the primary amine group and the second reactive moiety may comprise the cyclic amide ester group.

FIG. 6B shows the formation of a cyclooctatriazole covalent linkage 624 through a strain-promoted azide-alkyne cycloaddition click chemistry (SPACC) reaction between a strained alkyne group, specifically, a (1R,8S,9s)-bicyclo[6.1.0]non-4-yn-9-yl-ester group 620 with an azide group 622. The box in FIG. 6B denotes where the new covalent bonding is formed. In this scheme, the first reactive moiety may comprise the strained alkyne group and the second reactive moiety may comprise the azide group, or the first reactive moiety may comprise the azide group and the second reactive moiety may comprise the strained alkyne group.

FIG. 6C shows the formation of a cyclooctapyridazine covalent linkage 634 through a strain-promoted tetrazine ligation coupling reaction between a tetrazine group, specifically, a 1,2,4,5-tetrazin-3-yl group 630 and a strained alkene group, specifically, a cyclooct-4-en-1-yl-ester group 632. The box in FIG. 6C denotes where the new covalent bonding is formed. In this scheme, the first reactive moiety may comprise the strained alkene group and the second reactive moiety may comprise the tetrazine group, or the first reactive moiety may comprise the tetrazine group and the second reactive moiety may comprise the strained alkene group.

In some embodiments, the present disclosure provides hydrogels that comprise a crosslinked reaction product of a reactive polymer comprising first reactive moieties that comprise electrophilic groups and a multifunctional compound comprising second reactive moieties that comprise nucleophilic groups, wherein a dye molecule is covalently attached to the crosslinked reaction product. In some embodiments, the present disclosure provides hydrogels that comprise a crosslinked reaction product of a reactive polymer comprising first reactive moieties that comprise nucleophilic groups and a multifunctional compound comprising second reactive moieties that comprise electrophilic groups, wherein a dye molecule is covalently attached to the crosslinked reaction product.

In some embodiments, the present disclosure provides a system for forming hydrogels by combining the following in a reaction mixture: a reactive polymer comprising first reactive moieties that comprise electrophilic groups, and a multifunctional compound comprising second reactive moieties that comprise nucleophilic groups, wherein a dye molecule is covalently attached to the reactive polymer, the reactive multifunctional compound, or both. In some embodiments, the present disclosure provides a system for forming hydrogels by combining the following in a reaction mixture: a reactive polymer comprising first reactive moieties that comprise nucleophilic groups, and a multifunctional compound comprising second reactive moieties that comprise electrophilic groups, wherein a dye molecule is covalently attached to the reactive polymer, the reactive multifunctional compound, or both. The reactive polymer and the multifunctional compound are combined under conditions such that the electrophilic and nucleophilic groups crosslink with one another, forming the hydrogel, wherein the dye molecule is covalently attached to the hydrogel. In certain embodiments, those conditions comprise an environment having a basic pH, for example, a pH ranging from about 9 to about 11. Such hydrogels can be formed in vivo or ex vivo.

In some embodiments, the present disclosure provides hydrogels that comprise a crosslinked reaction product of a reactive polymer comprising first reactive moieties that comprise strained alkyne groups and a multifunctional compound comprising second reactive moieties that comprise azide groups, wherein a dye molecule is covalently attached to the crosslinked reaction product. In some embodiments, the present disclosure provides hydrogels that comprise a crosslinked reaction product of a reactive polymer comprising first reactive moieties that comprise azide groups and a multifunctional compound comprising second reactive moieties that comprise strained alkyne groups, wherein a dye molecule is covalently attached to the crosslinked reaction product.

In some embodiments, the present disclosure provides a system for forming hydrogels by combining the following in a reaction mixture: a reactive polymer comprising first reactive moieties that comprise strained alkyne groups, and a multifunctional compound comprising second reactive moieties that comprise azide groups, wherein a dye molecule is covalently attached to the reactive polymer, the reactive multifunctional compound, or both. In some embodiments, the present disclosure provides a system for forming hydrogels by combining the following in a reaction mixture: a reactive polymer comprising first reactive moieties that comprise azide groups, and a multifunctional compound comprising second reactive moieties that comprise strained alkyne groups, wherein a dye molecule is covalently attached to the reactive polymer, the reactive multifunctional compound, or both. The reactive polymer and the multifunctional compound are combined under conditions such that the strained alkyne and azide groups crosslink with one another, forming the hydrogel, wherein the dye molecule is covalently attached to the hydrogel. Such hydrogels can be formed in vivo or ex vivo.

In some embodiments, the present disclosure provides hydrogels that comprise a crosslinked reaction product of a reactive polymer comprising first reactive moieties that comprise strained alkyne groups and a multifunctional compound comprising second reactive moieties that comprise tetrazine groups, wherein a dye molecule is covalently attached to the crosslinked reaction product. In some embodiments, the present disclosure provides hydrogels that comprise a crosslinked reaction product of a reactive polymer comprising first reactive moieties that comprise tetrazine groups and a multifunctional compound comprising second reactive moieties that comprise strained alkyne groups, wherein a dye molecule is covalently attached to the crosslinked reaction product.

In some embodiments, the present disclosure provides a system for forming hydrogels that comprise a crosslinked reaction product by combining the following in a reaction mixture: a reactive polymer comprising first reactive moieties that comprise strained alkyne groups, and a multifunctional compound comprising second reactive moieties that comprise tetrazine groups, wherein a dye molecule is covalently attached to the reactive polymer, the reactive multifunctional compound, or both. In some embodiments, the present disclosure provides a system for forming hydrogels that comprise a crosslinked reaction product by combining the following in a reaction mixture: a reactive polymer comprising first reactive moieties that comprise tetrazine groups, and a multifunctional compound comprising second reactive moieties that comprise strained alkyne groups, wherein a dye molecule is covalently attached to the reactive polymer, the reactive multifunctional compound, or both. The reactive polymer and the multifunctional compound are combined under conditions such that the strained alkyne and tetrazine groups crosslink with one another, forming the hydrogel, wherein the dye molecule is covalently attached to the hydrogel. Such hydrogels can be formed in vivo or ex vivo.

In various embodiments, the hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values) for example, when measured on a bench-top micro CT system such as X treme CT from Scanco Medical (Wangen-Brüttisellen, Switzerland) or similar.

The hydrogels of the present disclosure can be used in a wide variety of biomedical applications, including implants, medical devices, and pharmaceutical compositions.

Various example of systems in accordance with the present disclosure that comprise a reactive polymer and a reactive multifunctional compound are described below. While not always explicitly stated, in these systems, a dye molecule is covalently attached to the reactive polymer, the reactive multifunctional compound, or both.

In some aspects of the present disclosure, a system is provided that comprises (a) a first composition that comprises a reactive polymer as described herein and (b) a second composition that comprises multifunctional compound as described herein, wherein when the first and second compositions are combined, covalent crosslinks form between the reactive polymer and the multifunctional compound, creating a hydrogel, wherein the dye molecule is covalently attached to the hydrogel.

The first composition may be a first fluid composition comprising the multifunctional compound or a first dry composition that comprises the multifunctional compound, to which a suitable fluid such as water for injection, saline, etc. can be added to form a first fluid composition. In addition to the multifunctional compound, the first composition may further comprise additional agents, including therapeutic agents, imaging agents, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, tonicity adjusting agents, suspension agents, wetting agents, and pH adjusting agents as described below.

In some embodiments, the system is configured to combine a first fluid composition comprising the multifunctional compound with a second fluid comprising the reactive polymer, wherein the first composition, the second composition, or both, comprises covalently attached dye molecule as described herein, and wherein the first composition, the second composition, or both, may optionally comprise additional agents as described herein. Upon mixing the first and second fluid compositions, the multifunctional compound crosslink with the reactive polymer, forming a hydrogel, wherein the dye molecule is covalently attached to the hydrogel. The first and second fluid compositions may be combined to form dye-containing crosslinked hydrogels, either in vivo or ex vivo.

In some embodiments, the multifunctional compound is initially combined with the reactive polymer under conditions where crosslinking between the reactive polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multifunctional compound and the reactive polymer, thereby forming a crosslinked product, wherein the dye molecule is covalently attached to the crosslinked product.

In some embodiments, the system comprises (a) a first composition that comprises a multifunctional compound as described herein and a reactive polymer as described herein and (b) a second composition, specifically, an accelerant composition, that contains an accelerant that is configured to accelerate a crosslinking reaction between the multifunctional compound and the reactive polymer. In some embodiments, the first composition may further comprise additional agents as described herein. In some embodiments, the second composition may further comprise additional agents as described herein.

In some embodiments, the system comprises (a) a first composition that comprises multifunctional compound as described herein, (b) a second composition that comprises a reactive polymer as described herein, and (c) a third composition, specifically, an accelerant composition, that contains an accelerant that is configured to accelerate a crosslinking reaction between the multifunctional compound and the reactive polymer. In some embodiments, the first composition may further comprise additional agents as described herein. In some embodiments, the second composition may further comprise additional agents as described herein. In some embodiments, the third composition may further comprise additional agents as described herein.

The first composition may be a first fluid composition comprising the multifunctional compound that is buffered to an acidic pH or a first dry composition that comprises the multifunctional compound, to which a suitable fluid such as water for injection, saline, an acidic buffer solution, etc. can be added to form a first fluid composition comprising the multifunctional compound that is buffered to an acidic pH. In some embodiments, for example, the acidic buffering composition may comprise monobasic sodium phosphate, among other possibilities. The first fluid composition comprising the multifunctional compound may have a pH ranging, for example, from about 3 to about 5. In addition to the multifunctional compound, the first composition may further comprise additional agents, including therapeutic agents, imaging agents, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the multifunctional compound that is buffered to an acidic pH. In addition to the reactive polymer, the second composition may further comprise additional agents, including therapeutic agents, imaging agents, tonicity adjusting agents, suspension agents, wetting agents, and pH adjusting agents as described below.

In a particular embodiment, the first composition is a first fluid composition comprising the multifunctional compound (as well as additional agents in some cases) that is buffered to an acidic pH and the second composition is a dry composition that comprises the reactive polymer (as well as additional agents in some cases). The first composition may then be mixed with the second composition to provide a prepared fluid composition that is buffered to an acidic pH and comprises the multifunctional compound and the reactive polymer. In a particular example, a syringe may be provided that contains the first fluid composition comprising the multifunctional compound that is buffered to an acidic pH, and a vial may be provided that comprises the second dry composition (e.g., a powder) that comprises the reactive polymer. The syringe may then be used to inject the first fluid composition into the vial containing the reactive polymer to form a prepared fluid composition that is buffered to an acidic pH and contains the multifunctional compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the multifunctional compound and the reactive polymer as described above (as well as additional agents in some cases), and a fluid accelerant composition that is buffered to basic pH as described above (which may include additional agents in some cases), may be combined form dye-containing crosslinked hydrogels, either in vivo or ex vivo.

Additional agents for use in the compositions described herein include therapeutic agents, imaging agents, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises multifunctional compound as described above and a second reservoir that contains a second fluid composition that comprises a reactive polymer as described above, wherein the first and second fluid compositions form a crosslinked product upon mixing. As previously noted, the first fluid composition, the second fluid composition, or both, may optionally comprise additional agents as described herein.

In some embodiments, the system may include a delivery device that comprises a first reservoir that contains a first fluid composition that comprises the multifunctional compound and the reactive polymer and is buffered to an acidic pH, such as the prepared fluid composition previously described, and a second reservoir that contains second fluid composition, such as the fluid accelerant composition previously described. As previously noted, the first fluid composition, the second fluid composition, or both, may optionally comprise additional agents as described herein.

In either case, during operation, the first fluid composition and second fluid composition are dispensed from the first and second reservoirs and combined, whereupon the multifunctional compound and the reactive polymer and crosslink with one another to form a dye-containing crosslinked hydrogel.

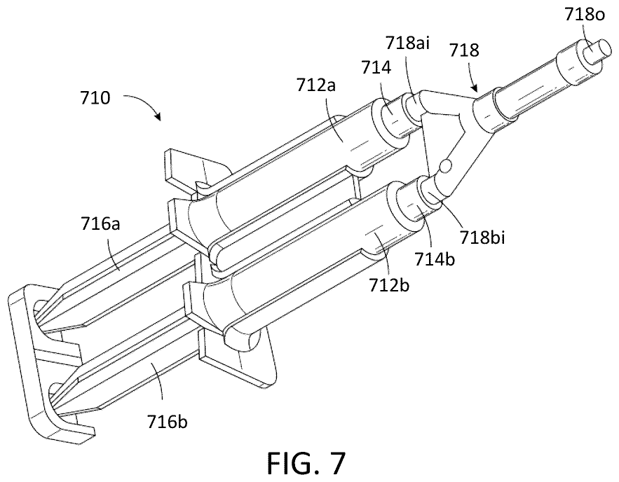

In particular embodiments, and with reference to FIG. 7, the system may include a delivery device 710 that comprises a double-barrel syringe, which includes first barrel 712a having a first barrel outlet 714a, which first barrel contains the first composition, a first plunger 716a that is movable in the first barrel 712a, a second barrel 712b having a second barrel outlet 714b, which second barrel 712b contains the second composition, and a second plunger 716b that is movable in the second barrel 712b. In some embodiments, the device 710 may further comprise a mixing section 718 having a first mixing section inlet 718ai in fluid communication with the first barrel outlet 714a, a second mixing section inlet 718bi in fluid communication with the second barrel outlet, and a mixing section outlet 718o.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a dye-containing crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a dye-containing crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for A sherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, dye-containing crosslinked hydrogels may be in any desired form, including a slab, a cylinder, a coating, or a particle. In some embodiments, the dye-containing crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Dye-containing crosslinked hydrogel particles formed using the above and other techniques may varying widely in size, for example, having an average size ranging from 50 to 950 microns.

In addition to a dye-containing crosslinked hydrogel as described above, dye-containing crosslinked hydrogel compositions in accordance with the present disclosure may contain additional agents, including therapeutic agents, imaging agents, tonicity adjusting agents, suspension agents, wetting agents, and pH adjusting agents as described above.

In various embodiments, kits are provided that include one or more delivery devices for delivering the dye-containing crosslinked hydrogel to a subject. Such systems may include one or more of the following: a syringe barrel, which may or may not contain a dye-containing crosslinked hydrogel as described herein; a vial, which may or may not contain a dye-containing crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the dye-containing crosslinked hydrogel may be provided in dry form (e.g., powder form) or in a form that is ready for injection, such as an injectable hydrogel form (e.g., a suspension of dye-containing crosslinked hydrogel particles).

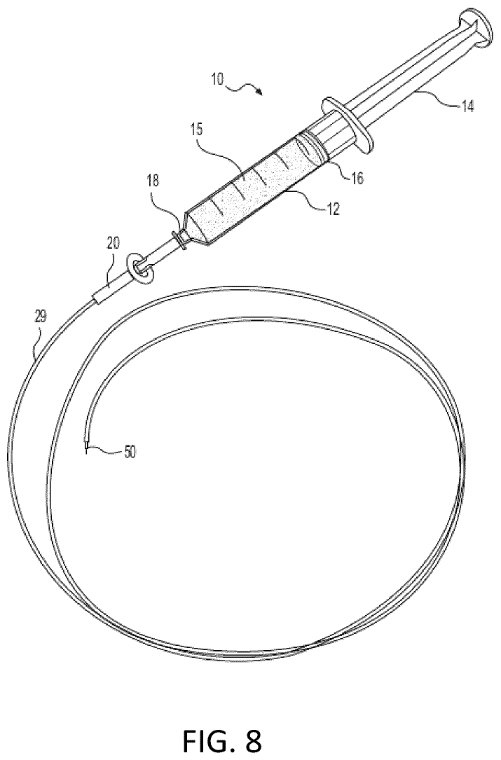

FIG. 8 illustrates a syringe 10 providing a reservoir for a dye-containing crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a dye-containing crosslinked hydrogel composition 15 for injection through the needle 50.

The dye-containing crosslinked hydrogel compositions described herein can be used for a number of purposes.

For example, dye-containing crosslinked hydrogel compositions can be injected to provide spacing between tissues, dye-containing crosslinked hydrogel compositions can be injected (e.g., in the form of blebs) to provide fiducial markers, dye-containing crosslinked hydrogel compositions can be injected for tissue augmentation or regeneration, dye-containing crosslinked hydrogel compositions can be injected as a filler or replacement for soft tissue, dye-containing crosslinked hydrogel compositions can be injected to provide mechanical support for compromised tissue, dye-containing crosslinked hydrogel compositions be injected as a scaffold, and/or dye-containing crosslinked hydrogel compositions can be injected as a carrier of therapeutic agents in the treatment of diseases and cancers and the repair and regeneration of tissue, among other uses.

During and/or after administration, the dye-containing crosslinked hydrogel compositions of the present disclosure can be imaged using a suitable imaging technique.

As seen from the above, the dye-containing crosslinked hydrogel compositions of the present disclosure may be used in a variety of medical procedures, including the following, among others: a procedure to implant a fiducial marker comprising a dye-containing crosslinked hydrogel, a procedure to implant a tissue regeneration scaffold comprising a dye-containing crosslinked hydrogel, a procedure to implant a tissue support comprising a dye-containing crosslinked hydrogel, a procedure to implant a tissue bulking agent comprising a dye-containing crosslinked hydrogel, a procedure to implant a therapeutic-agent-containing depot comprising a dye-containing crosslinked hydrogel, a tissue augmentation procedure comprising implanting a dye-containing crosslinked hydrogel, a procedure to introduce a dye-containing crosslinked hydrogel between a first tissue and a second tissue to space the first tissue from the second tissue.

The dye-containing crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for A sherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Dye-containing crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system comprising (a) one or more reactive multi-arm polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule.

2. The system of claim 1, wherein the system comprises a plurality of the one or more reactive multi-arm polymers, wherein a first fraction of the plurality of the one or more reactive multi-arm polymers comprise the covalently attached dye molecule, and wherein a second fraction of the plurality of the one or more reactive multi-arm polymers do not comprise the covalently attached dye molecule.

3. The system of claim 2, wherein the covalently attached dye molecule is covalently attached to the first fraction of the plurality of the one or more reactive multi-arm polymers through an amide group, wherein the covalently attached dye molecule is covalently attached to the first fraction of the plurality of the one or more reactive multi-arm polymers through an ester group, wherein the covalently attached dye molecule is covalently attached to the first fraction of the plurality of the one or more reactive multi-arm polymers through a triazine group, wherein the covalently attached dye molecule is covalently attached to the first fraction of the plurality of the one or more reactive multi-arm polymers through an amine group, or wherein the covalently attached dye molecule is covalently attached to the first fraction of the plurality of the one or more reactive multi-arm polymers through an ether group.

4. The system of claim 2, wherein the covalently attached dye molecule is covalently attached to the first fraction of the plurality of the one or more reactive multi-arm polymers through an amide group and an ester group, wherein the covalently attached dye molecule is covalently attached to the first fraction of the plurality of the one or more reactive multi-arm polymers through two ester groups, or wherein the covalently attached dye molecule is covalently attached to the first fraction of the plurality of the one or more reactive multi-arm polymers through a triazine group and an ester group.

5. The system of claim 2, wherein the first fraction ranges from 0.00001 mol % to 0.1 mol % and the second fraction ranges from 99.9 mol % to 99.99999 mol %, or wherein the weight of the first fraction ranges from 0.1 ppm to 1000 ppm of the total weight of the first and second fractions.

6. The system of claim 1, wherein the system comprises a plurality of the one or more reactive multifunctional compounds, wherein a first fraction of the plurality of the one or more reactive multifunctional compounds comprise the covalently attached dye molecule, and wherein a second fraction of the plurality of the one or more reactive multifunctional compounds do not comprise the covalently attached dye molecule.

7. The system of claim 6, wherein the covalently attached dye molecule is covalently attached to the first fraction of the plurality of the one or more reactive multifunctional compounds through an amide group, wherein the covalently attached dye molecule is covalently attached to the first fraction of the plurality of the one or more reactive multifunctional compounds through an ester group, wherein the covalently attached dye molecule is covalently attached to the first fraction of the plurality of the one or more reactive multifunctional compounds through a triazine group, wherein the covalently attached dye molecule is covalently attached to the first fraction of the plurality of the one or more reactive multifunctional compounds through an amine group, or wherein the covalently attached dye molecule is covalently attached to the first fraction of the plurality of the one or more reactive multifunctional compounds through an ether group.

8. The system of claim 6, wherein the covalently attached dye molecule is covalently attached to the first fraction of the plurality of the one or more reactive multifunctional compounds through an amide group and an ester group, wherein the covalently attached dye molecule is covalently attached to the first fraction of the plurality of the one or more reactive multifunctional compounds through two ester groups, or wherein the covalently attached dye molecule is covalently attached to the first fraction of the plurality of the one or more reactive multifunctional compounds through a triazine group and an ester group.

9. The system of claim 6, wherein the first fraction ranges from 0.00001 mol % to 0.1 mol % and the second fraction ranges from 99.9 mol % to 99.99999 mol %, or wherein the weight of the first fraction ranges from 0.1 ppm to 1000 ppm of the total weight of the first and second fractions.

10. The system of claim 1, wherein the hydrophilic polymer segments are selected from poly(alkylene oxide) segments, polysaccharide segments, polyoxazoline segments, polydioxanone segments, polypeptide segments, and polyvinyl alcohol segments.

11. The system of claim 1, wherein the plurality of first reactive moieties comprise a cyclic imide ester group and the plurality of second reactive moieties comprise a primary amine, thiol or hydroxyl group, or wherein the plurality of first reactive moieties comprise a primary amine, thiol or hydroxyl group and the plurality of second reactive moieties comprise a cyclic imide ester group.

12. The system of claim 1, wherein the plurality of first reactive moieties comprise a strained alkyne group and the plurality of second reactive moieties comprise an azide group, or wherein the plurality of first reactive moieties comprise an azide group and the plurality of second reactive moieties comprise a strained alkyne group.

13. The system of claim 1, wherein the plurality of first reactive moieties comprise a strained alkene group and the plurality of second reactive moieties comprise a tetrazine group, or wherein the plurality of first reactive moieties comprise a tetrazine group and the plurality of second reactive moieties comprise a strained alkene group.

14. The system of claim 1, further comprising a delivery device.

15. A method of treatment comprising administering to a subject a mixture that comprises (a) one or more reactive multi-arm polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule under conditions such that the one or more reactive multi-arm polymers and the one or more reactive multifunctional compounds covalently crosslink after administration to form a hydrogel, wherein the covalently attached dye molecule is covalently attached to the hydrogel.

16. The method of claim 15, wherein the method comprises administering to the subject a first fluid composition that comprises the one or more reactive multi-arm polymers and a second fluid composition that comprises the one or more reactive multifunctional compounds.

17. The method of claim 15, wherein the method comprises administering to the subject a first fluid composition that comprises the one or more reactive multi-arm polymers and the one or more reactive multifunctional compounds and a second fluid composition that comprises an accelerant that accelerates formation of covalent crosslinks.

18. A dye-containing hydrogel composition comprising a crosslinked reaction product of (a) one or more reactive multi-arm polymers that comprises three or more polymer arms linked to a core region, each arm comprising a hydrophilic polymer segment and a first reactive moiety, (b) one or more reactive multifunctional compounds comprising a plurality of second reactive moieties that are reactive with the first reactive moieties, and (c) a covalently attached dye molecule, wherein the covalently attached dye molecule is covalently attached to the crosslinked reaction product.

19. The dye-containing hydrogel composition of claim 18, wherein the dye-containing hydrogel is in the form of injectable particles.

20. A method of treatment comprising administering to a subject the dye-containing hydrogel composition of claim 18.
